(19) United States
(12) Patent Application Publication
(10) Pub. No.: US 2019/0385199 A1
(43) Pub. Date: Dec. 19, 2019

(54) REVIEW AND RECOMMENDATION FILTERING BASED ON USER FITNESS METRIC

(71) Applicant: International Business Machines Corporation, Armonk, NY (US)

(72) Inventors: Michael Bender, Rye Brook, NY (US); Rhonda L. Childress, Austin, TX (US); Kim A. Eckert, Austin, TX (US); Minh Q. Pham, Austin, TX (US)

(21) Appl. No.: 16/010,598

(22) Filed: Jun. 18, 2018

(51) Int. Cl. G06Q 30/02; G16H 20/30; G06F 17/30; G06Q 30/06

(52) U.S. Cl. CPC G06Q 30/0282; G06Q 30/0631; G06F 17/30867; G16H 20/30

(57) ABSTRACT

A system and method for filtering recommendations for an exercise machine based on a user fitness metric compared to similar fitness metrics of other users includes receiving biometric and activity data from a plurality of registered users, obtaining a machine usage data from a plurality of internet-connected exercise machines associated with the plurality of registered users, profiling the plurality of registered users using the biometric and activity data and the machine usage data to determine a fitness metric of each registered user, calculating the user fitness metric, in response to receiving a user request for recommendations and reviews for the exercise machine, and filtering the recommendations and reviews for the exercise machine to exclude recommendations and reviews from the plurality of registered users having a fitness metric that does not correspond to the user fitness metric.

### TECHNICAL FIELD

The present invention relates to systems and methods for filtering reviews and recommendations for exercise machines, and more specifically the embodiments of a filtering system for filtering recommendations for an exercise machine based on a user fitness metric compared to similar fitness metrics of other users.

### BACKGROUND

Consumers often look to reviews and recommendations when purchasing exercise equipment. The reviews and recommendations available on the Internet are authored by people of various fitness levels, conditioning levels, weights, and abilities. However, the consumer's physiological shape and fitness conditioning can impact whether a particular exercise machine is ideal for the consumer, rendering a particular review or recommendation irrelevant to the consumer.

### SUMMARY

An embodiment of the present invention relates to a method, and associated computer system and computer program product, for filtering recommendations for an exercise machine based on a user fitness metric compared to similar fitness metrics of other users. A processor of a computing system receives biometric and activity data from a plurality of registered users, the biometric and activity data being transmitted from one or more wearable computing devices worn by the plurality of registered users. A machine usage data is obtains from a plurality of internet-connected exercise machines associated with the plurality of registered users. The plurality of registered users are profiled using the biometric and activity data and the machine usage data to determine a fitness metric of each registered user of the plurality of registered users. The user fitness metric is calculated, in response to receiving a user request for recommendations and reviews for the exercise machine, wherein the user fitness metric is calculated based on a user biometric and activity data transmitted by at least one of a user mobile device and a user wearable device. The recommendations and reviews posted by the plurality of registered users relevant to the exercise machine are retrieved. The recommendations and reviews for the exercise machine are filtered to exclude recommendations and reviews from the plurality of registered users having a fitness metric that does not correspond to the user fitness metric, such that a website displaying the recommendations and reviews is altered so that the user is unable to view excluded recommendations and reviews for the exercise machine.

### BRIEF DESCRIPTION OF THE DRAWINGS

FIG. 1 depicts a block diagram of a filtering system, in accordance with embodiments of the present invention.

FIG. 2 depicts a table of calculated total fitness metric scores for registered users, in accordance with embodiments of the present invention.

FIG. 3 depicts a graphical user interface (GUI) of the user device showing unfiltered search results from a search query for an exercise machine, in accordance with embodiments of the present invention.

FIG. 4 depicts a table of registered users that have authored relevant reviews to the exercise machine being searched by the user and filtered based on the user fitness metric, in accordance with embodiments of the present invention.

FIG. 5 depicts a modified graphical user interface of the user device of FIG. 3 showing filtered search results from the search query for the exercise machine, in accordance with embodiments of the present invention.

FIG. 6 depicts a table of Registered Users that have authored relevant reviews to the exercise machine being searched by the user and filtered based on the user fitness metric, in accordance with embodiments of the present invention.

FIG. 7 depicts a modified graphical user interface of the user device of FIG. 3 showing filtered search results from the search query for the exercise machine, in accordance with embodiments of the present invention.

FIG. 8 depicts a flow chart of a method for filtering recommendations for an exercise machine based on a user fitness metric compared to similar fitness metrics of other users, in accordance with embodiments of the present invention.

FIG. 9 depicts a flow chart of a step of the method of FIG. 8 for filtering recommendations for an exercise machine based on a user fitness metric compared to similar fitness metrics of other users, in accordance with embodiments of the present invention.

FIG. 10 depicts a block diagram of a computer system for the filtering system of FIGS. 1-7, capable of implementing for filtering recommendations for an exercise machine based on a user fitness metric compared to similar fitness metrics of other users of FIGS. 8-9, in accordance with embodiments of the present invention.

FIG. 11 depicts a cloud computing environment, in accordance with embodiments of the present invention.

FIG. 12 depicts abstraction model layers, in accordance with embodiments of the present invention.

### DETAILED DESCRIPTION

Exercise equipment, exercise machines, and exercise accessories have various capabilities targeting specific fitness movements, with varying levels of difficulty. To help a user decide which exercise machine to purchase, the user often researches the exercise machine by reading online reviews and recommendations posted to various websites. However, the online reviews and recommendations are authored by people of varying levels of fitness and conditioning ability, lifestyles, weights, ages, gender, health, etc. Thus, relying on random and potentially irrelevant online reviews and recommendations for a particular user seeking to purchase exercise equipment can be problematic. Embodiments of the present invention may bridge the gap between searching for an ideal exercise machine in the market and relevant reviews and recommendations from members of a fitness community that share a similar fitness level as the user. As a result, an online review/recommendation filtering system may rank exercise machines available in a marketplace for display on a website such as search engine results, relative to user's fitness level at the time of purchasing an exercise machine. The filtering system may filter recommendations and/or make recommendations for exercise products based on a user's size, health, conditioning, etc. The users biometric and fitness/activity data may be provided by wearable computing devices, such as a smart watch, fitness bracelet, and the like. When searching for a new exercise machine product, the filtering system may compare a user's weight, heart rate, exercise frequency, and the like, to other peoples' weight, heart rate, exercise frequency, and the like. The filtering system may additionally analyze social media contacts and shared content to determine past purchases relating to the searched exercise machine. The reviews and recommendations from others who match the fitness level of the user may be displayed and ranked in order of relevancy, closeness to user fitness level, experience, etc.

Embodiments of the present invention may improve upon existing online reviews and recommendations platforms by filtering out reviews and recommendations from users that have a different fitness metric than the user searching/researching a particular exercise machine. By automatically filtering out results based on a fitness incompatibility reduces complication and ensures proper usability of the exercise machine for the user, based on the user's fitness level. Further, implementation of the present invention reduces product returns and disputes for a vendor and/or retailer. Improved search engine or website functionality may also provide recommendations, suggestion, allowing for upsell opportunities for additional compatible devices and similarly appropriate exercise machines and/or accessories.

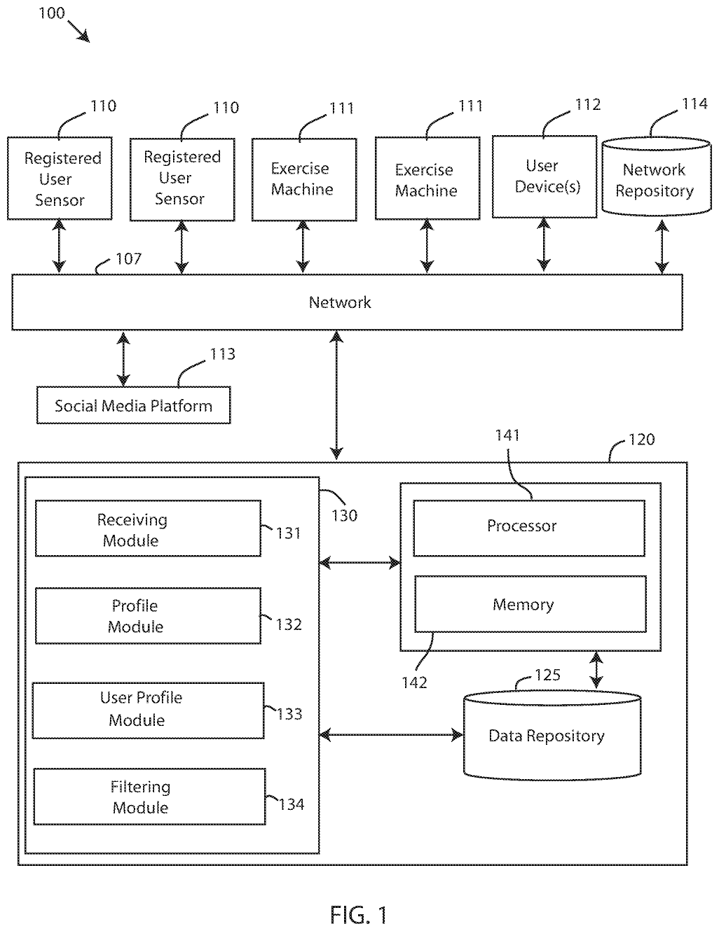

Referring to the drawings, FIG. 1 depicts a block diagram of a filtering system 100, in accordance with embodiments of the present invention. Embodiments of the filtering system 100 may be a system for filtering recommendations for an exercise machine based on a user fitness metric compared to similar fitness metrics of other users. Embodiments of the filtering system 100 may be useful for solving a problem relating to search engines and/or website online review/recommendation engines returning results for exercise machines written by users with a different fitness level than the user's fitness level, which results in misleading reviews from the perspective of the user considering a purchase of an exercise machine. For example, the filtering system 100 may tailor/filter/individualize online reviews and/or recommendations regarding exercise equipment to the user based on a fitness level of the author of the review/recommendation that corresponds to a fitness level of the user, which may also be ordered in accordance with an optimum fit for a user's needs and existing fitness level. Embodiments of the filtering system 100 may also decrease a decision time for the user because the user no longer needs to research whether the author of the review/recommendation shares the same fitness level, age, weight, gender, etc., saving additional computer resources to further research the often private details of the authors writing about a particular exercise machine. Embodiments of an exercise machine may be any exercise of fitness based product that is intended to be used by a user for exercise or other health or fitness related benefit. Exemplary embodiments of exercise machine may be a treadmill, an elliptical machine, a stationary bicycle, a stair climber, a hybrid exercise device, a home gym system, a rower, a weight machine, and the like. Further, the filtering system 100 may also be applied to filter reviews/recommendations for exercise equipment, accessories, plans, programs, gyms, fitness mobile apps, streaming exercise channels, a digital video of an exercise program, a local gym, and the like, in a same or similar manner as applied to an exercise machine.

Embodiments of the filtering system 100 may be a search engine, a filter system, a search engine platform, a website searching functionality, an online review platform, an online recommendation platform, an lone product review and recommendation platform, a search results filtering system, a fitness based filtering system, a customizable product review system, and the like. Embodiments of the filtering system 100 may include a computing system 120. Embodiments of the computing system 120 may be a computer system, a computer, a server, one or more servers, a backend computing system, a search engine, a computing system powering a search engine, a computing system powering an online review/recommendation functionality of a website, and the like.

Furthermore, embodiments of filtering system 100 may include one or more registered user sensors 110, one or more exercise machines 111, a user device 112, and a social media platform 113 that are communicatively coupled to the computing system 120 over a network 107. For instance, information/data may be transmitted to and/or received from the one or more registered user sensors 110, the one or more exercise machines 111, the user device 112, and the social media platform 113 over a network 107. A network 107 may be the cloud. Further embodiments of network 107 may refer to a group of two or more computer systems linked together. Network 107 may be any type of computer network known by individuals skilled in the art. Examples of network 107 may include a LAN, WAN, campus area networks (CAN), home area networks (HAN), metropolitan area networks (MAN), an enterprise network, cloud computing network (either physical or virtual) e.g. the Internet, a cellular communication network such as GSM or CDMA network or a mobile communications data network. The architecture of the network 107 may be a peer-to-peer network in some embodiments, wherein in other embodiments, the network 107 may be organized as a client/server architecture.

In some embodiments, the network 107 may further comprise, in addition to the computing system 120, a connection to one or more network-accessible knowledge bases 114, which are network repositories containing information of exercise machines, user search activity, search engine filtering options, fitness metrics, network repositories or other systems connected to the network 107 that may be considered nodes of the network 107. In some embodiments, where the computing system 120 or network repositories allocate resources to be used by the other nodes of the network 107, the computing system 120 and network-accessible knowledge bases 114 may be referred to as servers.

The network-accessible knowledge bases 114 may be a data collection area on the network 107 which may back up and save all the data transmitted back and forth between the nodes of the network 107. For example, the network repository may be a data center saving and cataloging registered user data and exercise machine device/usage data, and the like, to generate both historical and predictive reports regarding a particular user fitness metric, device list, search query, product needs, and the like. In some embodiments, a data collection center housing the network-accessible knowledge bases 114 may include an analytic module capable of analyzing each piece of data being stored by the network-accessible knowledge bases 114. Further, the computing system 120 may be integrated with or as a part of the data collection center housing the network-accessible knowledge bases 114. In some alternative embodiments, the network-accessible knowledge bases 114 may be a local repository that is connected to the computing system 120.

Embodiments of the registered user sensors 110 may be one or more sensors 110 which may be positioned within an environment shared by the registered user, worn by the registered user, or otherwise disposed in a location that can result in obtaining registered user biometric and activity data. Sensors 110 may be a sensor, an input device, or any input mechanism. For example, sensor 110 may be a biometric sensor, a wearable sensor, an environmental sensor, a camera, a camcorder, a microphone, a peripheral device, a computing device, a mobile computing device, such as a smartphone or tablet, facial recognition sensor, voice capture device, and the like. Embodiments of sensors 110 may also include a heart rate monitor used to track a current or historical average heart rate of the registered user; wireless-enabled wearable technology, such as an activity tracker or smartwatch that tracks a heart rate, an activity level (e.g. number of calories burned, total steps in a day, etc.), a quality of sleep, a diet, a number of calories burned; a robotic therapeutic sensor; a blood pressure monitor; a perspiration sensor; and other wearable sensor hardware. Embodiments of sensors 110 may further include environmental sensors either worn or placed in a registered user environment, such as a home gym, that can measure air quality, temperature, pressure, NO₂ levels, humidity, and the like, which may be helpful in determining a fitness metric (e.g. fitness level, conditioning, athletic ability, biometrics, etc.). Further embodiments of sensor 110 not specifically listed herein may be utilized to collect data about the registered user or registered user fitness.

Further embodiments of sensors 110 may include one or more input devices or input mechanisms, including one or more cameras positioned proximate the registered user or within an environment shared by the registered user. The one or more cameras may capture image data or video data of a registered user exercising, including video data regarding perspiration, muscle activity, gestures, etc., while exercising. Further embodiments of sensors 110 may include a mobile computing device, such as a smartphone or tablet device, which may run various applications that contain fitness/activity data about the registered user. For example, an registered user's smartphone may include a fitness/health/activity application that may send exercise, fitness, activity, health, etc. data to the computing system 120, or may send relevant social media information to the computing system 120 via a social media application loaded on the user's smartphone. The mobile computing device as used as sensor 110 may also utilize the device's camera, microphone, and other embedded sensors to send information to the computing system 120. Moreover, embodiments of sensors 110 may encompass other input mechanisms, such as a user computer that may send information to the computing system 120, wherein the user computer may be loaded with software programs that are designed to track an exercise or activity or fitness output level. Embodiments of the sensor 110 may also transmit biometric information of each registered user, such as age, weight, gender, height, and the like.

Embodiments of a registered user may be a person that has registered with a central system supported by computing system 120, to transmit personal fitness information to the computing system. The user may register with the computing system 120 through one or more software applications, agreeing to offer biometric and activity information to the computing system to implement the online review and recommendation filtering system. Alternatively, the registered user may be a user that uses various software applications that automatically send biometric and activity information to the computing system 120 as part of the functioning of the various software application loaded on one or more computing devices, including one or more wearable devices (e.g. smartwatch, fitness tracker bracelet, etc.).

Referring still to FIG. 1, embodiments of the exercise machine 111 may be an exercise machine, device, equipment, sensor, and the like, which may connect to the Internet and provided machine usage data. For instance, embodiments of the exercise machine 111 may be an Internet-of-Things (IoT) device. Embodiments of the exercise machine 111 may include one or more sensors and associated network interface controllers that automatically send usage data and other data to the computing system 120 over network 107, when a registered user is using or when the registered user is not using the exercise machine 111. For example, the exercise machine 111 may transmit usage data, such as an average exercise time per session, a number of exercise sessions per day, per week, per months, etc., an intensity of each exercise session of the exercise machine 111 by the registered user, a type of exercise program used by the registered user when exercising, a period of inactivity between workouts, and the like. The exercise machine 111 may also automatically transmit identifying information, such as model number, date of manufacturing, a repair history, a maintenance record. Exemplary embodiments of exercise machine 111 may be an Internet connected treadmill, elliptical machine, stationary bicycle, stair climber, hybrid exercise device, home gym system, rower, weight machine, and the like. The machine usage data provided by the exercise machine 111 may be utilized to calculate a registered user fitness metric for a registered user associated with the exercise machine 111, as described in greater detail infra.

Embodiments of the user device 112 may be a computing device, a computer, a desktop computer, a cell phone, a mobile computing device, a tablet computer, a laptop computer, a wearable computing device, a smartwatch, and the like, which may be used to access and operate a search engine or website product search functionality using a browser loaded on the user device 112. The user device 112 may include hardware functionality such as a speaker for emitting a sound, a vibration motor for creating vibrations, a display for displaying images, videos, pictorial sequences, etc., a light emitting element for emitting a light, a receiver for receiving communications, a transmitter for transmitting signals, and other similar features and hardware of a computer, smartphone, smartwatch, cell phone, tablet computer, and the like.

Embodiments of the social media platform 113 may be one or more social media platforms including databases, storage devices, repositories, servers, computers, engines, and the like, that may service, run, store or otherwise contain information and/or data regarding a social media network of the user and the user's social contacts. The social media platform or platforms 113 may be accessed or may share a communication link over network 107, and may be managed and/or controlled by a third party, such as a social media company. In an exemplary embodiment, the social media platforms 113 may be a social media network, social media website, social media engine, and the like, which may store or otherwise contain content supplied by a social contact of the user.

Furthermore, embodiments of the computing system 120 of the filtering system 100 may be equipped with a memory device 142 which may store various data/information/code, and a processor 141 for implementing the tasks associated with the filtering system 100. In some embodiments, a product review/recommendation filtering application 130 may be loaded in the memory device 142 of the computing system 120. Embodiments of the product review/recommendation filtering application 130 may be an interface, an application, a program, a module, or a combination of modules. In an exemplary embodiment, the product review/recommendation filtering application 130 may be a software application running on one or more back end servers, search engines, etc. servicing one or more user devices 112, wherein a user interface portion of the software application (e.g. a search engine application, product website application, product review platform application) may also run on the user device 112.

The product review/recommendation filtering application 130 of the computing system 120 may include a receiving module 131, a profile module 132, a user profile module 133, and a filtering module 134. A “module” may refer to a hardware-based module, software-based module or a module may be a combination of hardware and software. Embodiments of hardware-based modules may include self-contained components such as chipsets, specialized circuitry and one or more memory devices, while a software-based module may be part of a program code or linked to the program code containing specific programmed instructions, which may be loaded in the memory device of the computing system 120. A module (whether hardware, software, or a combination thereof) may be designed to implement or execute one or more particular functions or routines.

Embodiments of the receiving module 131 may include one or more components of hardware and/or software program code for receiving biometric and activity data from a plurality of registered users. For instance, the biometric and activity data may be transmitted from one or more wearable computing devices and/or sensors 110 worn by or otherwise associated with the plurality of registered users. The sensors 110 associated with each registered user may communicate biometric data, such as an age of the person, a weight of the person, a gender of the person, a height of the person, a location of the person, and the like. The sensors 110 may also communicate activity data of each registered user. For example, the sensors 110 (e.g. sensors of a smartwatch running a fitness software application) may transmit an activity data of the user wearing the sensor 110, such as a current hear rate, an average heart rate, an average daily step total, a current step total, an average calories burned per day, an average daily exercise time, an average exercise intensity, average stand hours, an average walking distance over a given period of time, an average run time over a given period, a number of stair flights climbed over a given period of time, awards achieved by the user using a fitness application, average weight lifted for a given exercise, a longevity of activity, various historical reports of the user's activity, a heart rate variability, a resting heart rate, other vitals, and the like, or any data that may be useful in calculating a fitness metric of the registered user. The biometric and activity data of registered users may be received by the receiving module 131 of the computing system over network 107, and may organize and characterize the data according to each registered user in the system. Embodiments of the registered user may be a user registered within the system, or a user running an application that automatically sends biometric and activity the computing system 120. In further embodiments, the biometric and activity of each registered user may be sent to a third party server servicing one or more fitness applications on the registered user device, wherein the biometric and activity data is received from the third party server, in response to a request for the biometric and activity information of the registered user.

Moreover, embodiments of the receiving module 131 may receive, obtain, or otherwise collect a machine usage data from a plurality of internet-connected exercise machines 111 associated with the plurality of registered users. For instance, one or more exercise machine 111 belonging to the registered user may transmit machine usage data and machine identifying information to the computing system 120 to be used to calculate a registered user fitness metric. Embodiments of the machine usage data may be an average exercise time per session, a number of exercise sessions per day, per week, per months, etc., an intensity of each exercise session of the exercise machine 111 by the registered user, a type of exercise program used by the registered user when exercising, a period of inactivity between workouts, and the like. The machine usage data received by the receiving module 131 may be any data related to the operation of the exercise machine 111 by the registered user.

With continued reference to FIG. 1, embodiments of the computing system 120 may include a profile module 132. Embodiments of the profile module 132 may include one or more components of hardware and/or software program code for profiling the plurality of registered users using the biometric and activity data and the machine usage data to determine a fitness metric of each registered user of the plurality of registered users. For instance, embodiments of the profile module 132 may profile each registered user to calculate a fitness metric for each registered user. Embodiments of a fitness metric may be a measurement of a level of fitness, a fitness shape, a conditioning, a health, an activity, and the like, of a registered user. The fitness metric may be represented as a score or other numerical representation to quantify a fitness ability, status, level, etc. of a registered user. Embodiments of the fitness metric may be a combination of a biometric score, a machine data usage score, and potentially a social media content adjustment factor. The scores may be represented by a range of numbers (e.g. 1-400), wherein one end of the spectrum or range (e.g. 1) may represent a lower fitness level while the other end of the spectrum or range (e.g. 400) may represent a very high fitness level.

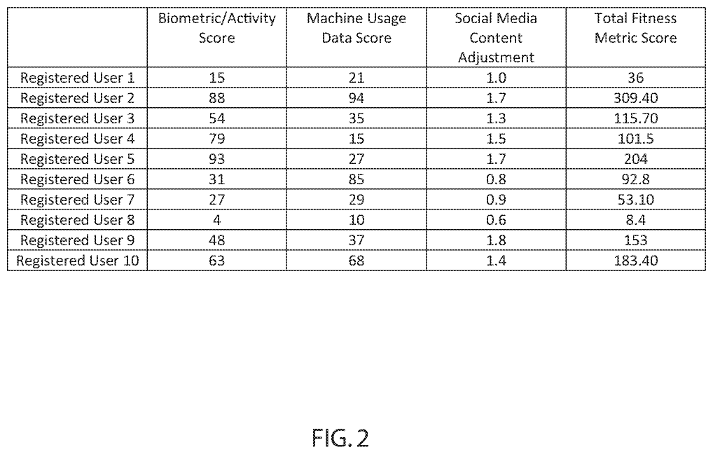

FIG. 2 depicts a table of calculated total fitness metric scores for registered users, in accordance with embodiments of the present invention. First, the profile module 132 may calculate a biometric/activity score for Registered Users 1-10, as shown in the second column. The biometric/activity score of the Registered Users 1-10 may be output as a numerical value, such as a metric score or rating. The biometric/activity score may be defined within a predefined range, such as 0-1, 0-10, 0-100, 0-200, 0-300, 0-400, and the like. In an exemplary embodiment, a higher fitness activity and fitness ability of the Registered User, the higher the biometric/activity score, as provided by the sensors 110 associated with the registered users. For instance, Registered Users 2 and 5 have the highest biometric/activity score, which means that registered Users 2 and 5 exercise more frequently at a higher intensity and/or longer duration than the other Registered Users. Other registered users, such as Registered Users 4 and 10 may have a lower biometric/activity score than Registered Users 2 and 5 because Registered Users 2 and 5 may exercise more frequently, with more intensity and/or more duration than Registered Users 4 and 10. The remaining Registered Users 1, 3, 6, 7, 8, and 9 may have a lower biometric/activity score because Registered Users 1, 3, 6, 7, 8, and 9 may exercise less frequently, with less intensity and/or less duration.

In an exemplary embodiment, the biometric/activity score is based on the data received by the sensors 110, such as a wearable computing device of the registered user. As a result, a portion of the fitness level may be attributable to exercise and activity of the registered user not involving a use of a particular exercise machine 111. In other words, a registered user (e.g. Registered User 5) may have a high biometric/activity score largely as a result from walking, running, hiking, etc. outdoors and not using a particular exercise machine 111, which may be a target of a user search for reviews and recommendations. Accordingly, the profile module 132 may calculate a machine usage score for Registered Users 1-10, as shown in the third column. The machine usage score of the Registered Users 1-10 may be output as a numerical value, such as a metric score or rating. The machine usage score may be defined within a predefined range, such as 0-1, 0-10, 0-100, 0-200, 0-300, 0-400, and the like. In an exemplary embodiment, the more the registered user uses the exercise machine, the longer the registered user uses the exercise machine 111, and/or the more work being performed onto the machine, the higher the machine usage score, as provided by the IoT sensor(s) associated with the exercise machine 111. For instance, Registered User 2 has a high biometric/activity score and the highest machine usage score, which indicates that Registered User 2 is very fit and uses the exercise machine 111 to obtain a high fitness level. Registered User 5, while having a high biometric/activity score, has a much lower machine usage score, which indicates that the user is very fit, but may not use the exercise machine 111 to largely obtain a high fitness level. The machine data usage score may be helpful to corroborate the data received from the sensors 110 of the registered users to improve an accuracy of the filtering system 100. Additionally, the machine usage score may be helpful to further profile a registered user's knowledge of a particular exercise machine, which may affect or impact an accuracy of the registered user's review or recommendation of the exercise machine. For example, if a user has a low machine usage score for a particular exercise machine, an accuracy or reliability of a review written by the registered user on the particular exercise machine may be adversely impacted, which in turn may affect a ranking of the registered user's review/recommendation, or may ultimately lead to the review/recommendation being filtered out from view of a user on a website.

Moreover, embodiments of the profile module 132 may apply a social media adjustment factor to a sum of the biometric/activity and machine usage scores of Registered Users 1-10. Alternatively, the social media adjustment factor may be applied to only one of the biometric/activity score or the machine usage score. The social media adjustment factor may be a numerical value representing an adjustment or multiplier for social media activity that corroborates the fitness level and/or machine usage of an exercise machine 111. The social media adjustment factor may be used to modify the fitness metric score (e.g. sum of biometric/activity score and machine usage score) to derive a total fitness metric score. For example, the social media content adjustment may be multiplied with the sum of biometric/activity score and machine usage score.

Embodiments of the profile module 132 may monitor, analyze, evaluate, etc. a shared social media content of the registered user to determine if the registered user has shared/posted content on one or more social media platforms 113 that is relevant to the registered user's fitness/activity or use of the exercise machine. For instance, embodiments of the profile module 132 may include one or more components of hardware and/or software program code for analyzing a content shared by the registered user on one or more social media platforms 113 to determine that a context of a content shared correlates to exercise, fitness, healthy lifestyle, exercise machines, etc. The shared content shared, uploaded, or otherwise posted on the social media platform 113 may be photographs, videos, comments made on other contacts' pages, text-based posts made to the social contact's own social media page, and the like. The shared content may be analyzed, parsed, scanned, searched, inspected, etc. for a context that correlates or otherwise relates to or is associated with exercise, fitness, healthy lifestyle, exercise machines, etc. In an exemplary embodiment, the profile module 132 may utilize a natural language technique to determine keywords associated with the content available on the social media platform 113, and then examine the determined keywords with keywords that may be relatable with the exercise, fitness, healthy lifestyle, exercise machines, etc. In another exemplary embodiment, the profile module 132 may utilize an image or visual recognition engine to inspect, parse, scan, analyze, etc. a photograph, image, video, or other content to determine one or more descriptions or insights that describe or are associated with the photograph, image, video, or other content, and then examine the descriptions/insights with keywords that may be relatable with exercise, fitness, healthy lifestyle, exercise machines, etc. In yet another embodiment, the profile module 132 may use a combination of natural language techniques, cognitive applications/engines, and visual recognition engines to determine a context of the shared content available on the one or more social media platforms 113.

Moreover, embodiments of the profile module 132 may compare the determined context from the shared content with biometric activity data and machine usage data to determine whether the registered user's social media content further bolsters or detracts from the calculated score. For instance, if the registered user has shared a video of the registered user running on a treadmill, then the profile module 132 may calculate a social media content adjustment factor that may increase a total fitness metric score (e.g. Registered User 2). If the registered user has not shared content that includes fitness and activity, but has shared content suggesting that the registered user does not use an elliptical the user purchased ten months ago, then the profile module 132 may calculate a social media content adjustment factor that may decrease a total fitness metric score (e.g. Registered User 8). If the registered user does not have a social media account on the social media platform 113 or has made neutral comments or the shared content is irrelevant to a fitness level of the registered user, then the profile module 132 may not apply a social media content adjustment factor, or may apply a factor that neither increases or decreases the total fitness metric (e.g. Registered User 1).

Accordingly, embodiments of the profile module 132 may analyze a shared social media content of the plurality of registered users to augment the fitness metric of the plurality of registered users. The total fitness metric score may be stored on the computing system 120 or other database, for each registered user.

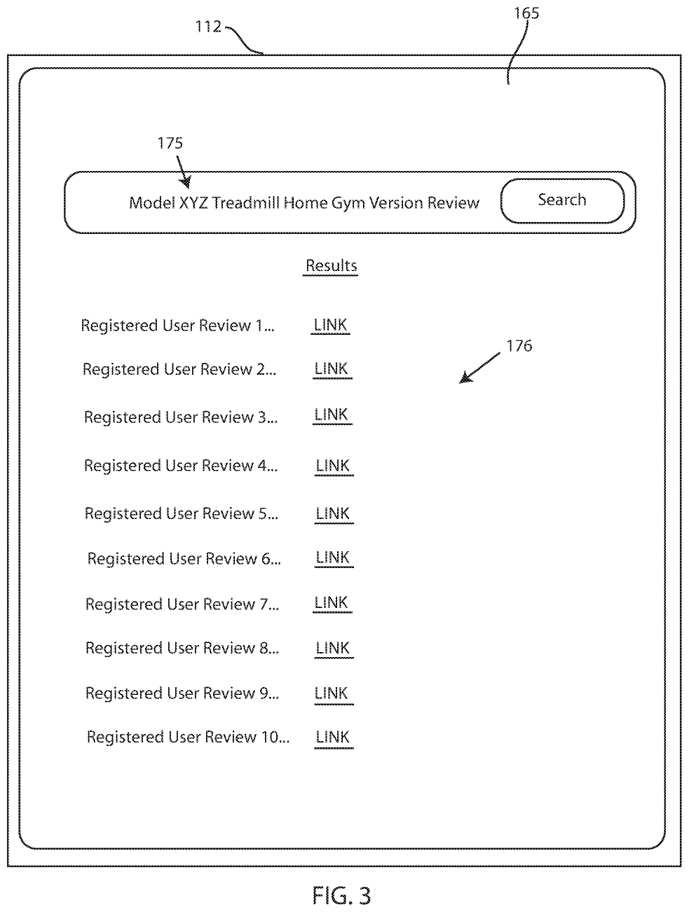

Referring back to FIG. 1, embodiments of the computing system 120 may include a user profile module 133. Embodiments of the user profile module 133 may include one or more components of hardware and/or software program code for calculating a user fitness metric, in response to receiving a user request for recommendations and reviews for a particular exercise machine. For instance, a user may input a search query for requesting online reviews and/or recommendations for a particular exercise machine that the user is considering purchasing. A graphical user interface of a search engine application loaded on the user device 112, or a website/webpage for product reviews may display the text as the user types or otherwise inputs (e.g. voice-to text, virtual assistant, etc.) the search query. In prior systems, the search query may be processed, and a number of search results may be displayed to the user as a function of the search engine processing the search query without filtering as described in greater detail infra. FIG. 3 depicts a graphical user interface (GUI) 165 of the user device 112 showing unfiltered search results 176 from a search query 175 for an exercise machine, in accordance with embodiments of the present invention. Here, the user has entered a search query 175 that states “Model XYZ Treadmill Home Gym Version Review.” Traditionally, a search engine would process the search query, fetch various websites, reviews, and recommendations, organize the websites with reviews according to search algorithms blind to the user's fitness level and fitness level of the authors of the reviews and recommendation, and then display the results 176 with links to the reviews written by registered users for the user to access the webpage, as shown in FIG. 3. However, as an improvement to existing search engine capabilities and review filtering and selection, the review/recommendation filtering application 130 may filter the reviews based on a match between the user fitness metric and the fitness metric of a registered user that has authored a review/recommendation in the unfiltered results 176.

Embodiments of the user fitness metric may be calculated based on a user biometric and activity data transmitted by at least one of a user mobile device and a user wearable device. For instance, embodiments of the user fitness metric may be a measurement of a level of fitness, a fitness shape, a conditioning, a health, an activity, and the like, of a user looking to purchase an exercise machine. The user fitness metric may be represented as a score or other numerical representation to quantify a fitness ability, status, level, etc. of the user. Embodiments of the user fitness metric may be a combination of a biometric/activity score and potentially a social media content adjustment factor. The scores may be represented by a range of numbers (e.g. 1-400), wherein one end of the spectrum or range (e.g. 1) may represent a lower fitness level while the other end of the spectrum or range (e.g. 400) may represent a very high fitness level. The biometric/activity score of the user may be output as a numerical value, such as a metric score or rating. The biometric/activity score may be defined within a predefined range, such as 0-1, 0-10, 0-100, 0-200, 0-300, 0-400, and the like. In an exemplary embodiment, a higher fitness activity and fitness ability of the user, the higher the biometric/activity score, as provided by the user device(s) 112, such as a wearable computing device of the user, or a fitness application running on the user mobile device. In an exemplary embodiment, the calculation of the user fitness metric may be performed in response to detection of the user search query 175. For example, the user profile module 133 may analyze the search query 175 input into a search engine or website search functionality to determine that the user is searching for reviews for an exercise machine. For instance, the user profile module 133 may, in response to detecting that a user is typing a search query into a search function user interface on the user device 112, or in real-time as the user is typing the search query, analyze a content of the search query to determine that the user is searching for reviews and recommendations for an exercise machine to purchase. For example, the user profile module 133 may parse the search query to determine which exercise machine the user is seeking reviews for to retrieve various online reviews and recommendations from registered users. The content of the search query may be analyzed by a text analysis system that may parse, identify, etc. words using, for example, a natural language processing technique, a natural language understanding technique, etc. to analyze the content (e.g. words) of the search query.

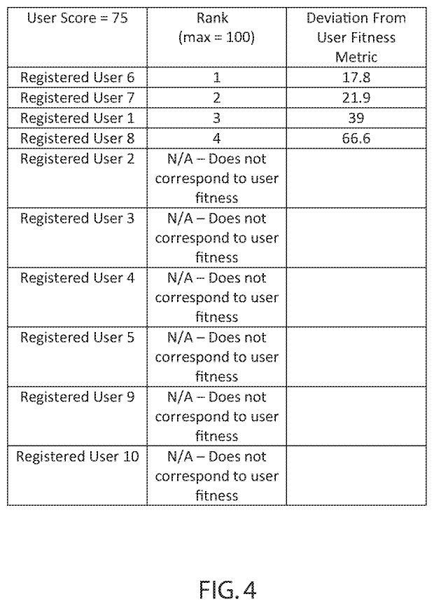

Embodiments of the computing system 120 may include a filtering module 134. Embodiments of the filtering module 134 may include one or more components of hardware and/or software program for retrieving the recommendations and reviews posted by the plurality of registered users relevant to the exercise machine, and filtering the recommendations and reviews for the exercise machine to exclude recommendations and reviews from the plurality of registered users having a fitness metric that does not correspond to the user fitness metric, such that a website displaying the recommendations and reviews is altered so that the user is unable to view excluded recommendations and reviews for the exercise machine. For instance, embodiments of the filtering module 134 may filter the search results/online reviews to take into account both the user fitness metric and the fitness metrics of the registered users that have authored a review for the exercise machine, such that the filtering module 134 may intentionally remove reviews, links, recommendations, etc. that are from a registered user whose total fitness metric score is not within a predetermined threshold or range of the user fitness metric. The range or threshold surrounding the user fitness metric score may vary, and if the fitness metric score for a registered user that authored a review showing in the unfiltered results is outside the range or threshold, the filtering module 134 may remove the result, link, reviews, etc. from even appearing on the webpage of the browser of the user device 112. FIG. 4 depicts a table of Registered Users that have authored relevant reviews to the exercise machine being searched by the user and filtered based on the user fitness metric, in accordance with embodiments of the present invention. As shown in FIG. 4, the filtering module 134 has filtered out Registered Users 2-5 and 9-10 because the fitness metric of the Registered Users 2-5 and 9-10 do not correspond to the user fitness metric score of 75; the Registered Users 2-5 and 9-10 have a fitness metric much higher than the user fitness metric. In other words, embodiments of the filtering module 134 may compare the user fitness metric to fitness metrics of the plurality of registered users that have posted a recommendation and review of the exercise machine, to determine whether the fitness metric of a registered user of the plurality of registered users corresponds to the user fitness metric. The fitness metric of the registered user may correspond to the user fitness metric if the fitness metric of the registered user is within a predetermined range of the user fitness metric. The reviews/recommendations associated with or authored by Registered Users 2-5 and 9-10 have been determined to not be reliable, relevant, effective, accurate, and/or appropriate for the user based on the disparity of fitness levels between the review author and the user, and are thus filtered out in a manner that the search engine/search platform running filtering application 130 may not display or otherwise present the links to the reviews or the reviews associated with Registered Users 2-5 and 9-10.

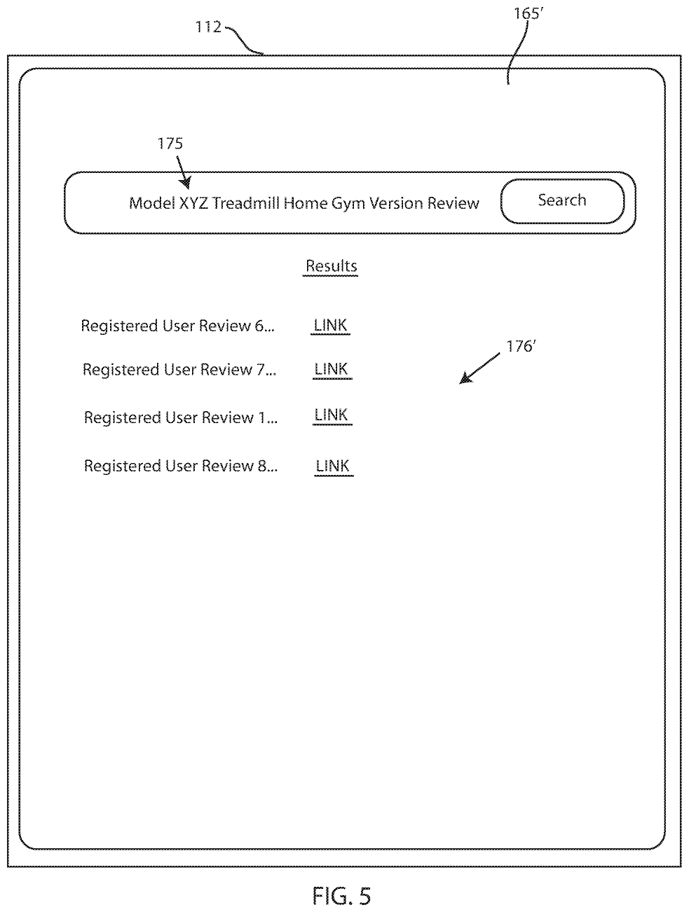

Rather, embodiments of the filtering module 134 may only display reviews or links to reviews that are associated with or authored by registered users having a fitness metric that corresponds to the user fitness metric, while removing reviews/recommendations from registered users having a fitness metric outside the acceptable threshold to the user fitness metric, so that user does not view the removed reviews. FIG. 5 depicts a modified graphical user interface 165′ of the user device 112 of FIG. 3 showing filtered search results 176′ from the search query 175 for the exercise machine, in accordance with embodiments of the present invention. As shown in FIG. 5, several reviews associated with registered users, or links to reviews of the exercise machine, have been filtered and removed so that the user is unable to select or visually see the links to the reviews written by registered users with an incompatible fitness level. Additionally, the reviews have been ranked and displayed in order of rank based on a lowest deviation of the total fitness metric from the user metric score, as calculated by the computing system 120. For example, Registered User Review 6 is displayed at the top of the results 176′ because the fitness metric score of Registered User 6 has the lowest deviation from the user metric score. Accordingly, reordering the reviews and removing the incompatible reviews may include augmenting a graphical user interface 165 of a user computing device 112 to change an appearance of the graphical user interface 165′ to improve a user purchase decision for user regarding an exercise machine.

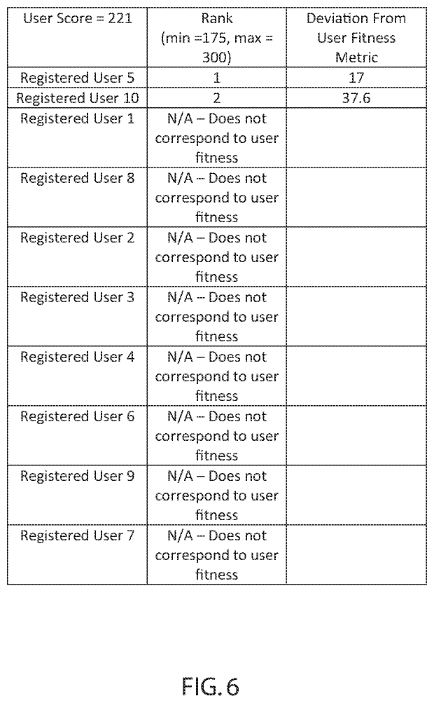

Similarly, FIG. 6 depicts a table of Registered Users that have authored relevant reviews to the exercise machine being searched by the user and filtered based on the user fitness metric, in accordance with embodiments of the present invention. As shown in FIG. 6, the filtering module 134 has filtered out Registered Users 1-4 and 6-9 because the fitness metric of the Registered Users 1-4 and 6-9 do not correspond to the user fitness metric score of 221; the Registered Users 1-4 and 6-9 have a fitness metric much lower than the user fitness metric. The reviews/recommendations associated with or authored by Registered Users 1-4 and 6-9 have been determined to not be reliable, relevant, effective, accurate, and/or appropriate for the user based on the disparity of fitness levels between the review author and the user, and are thus filtered out in a manner that the search engine/search platform running filtering application 130 may not display or otherwise present the links to the reviews or the reviews associated with Registered Users 1-4 and 6-9.

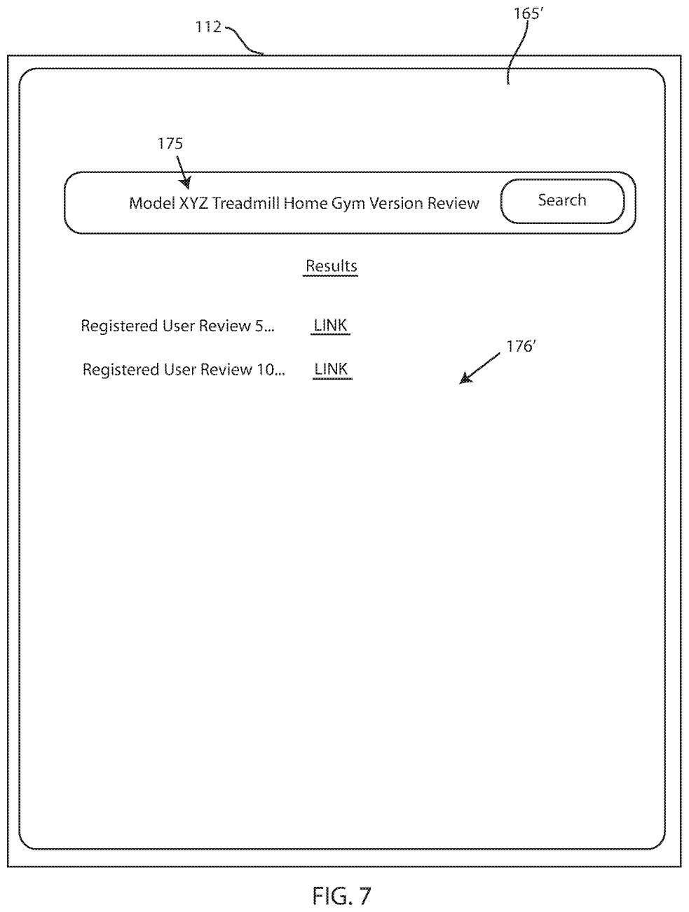

Rather, embodiments of the filtering module 134 may only display reviews or links to reviews that are associated with or authored by registered users having a fitness metric that corresponds to the user fitness metric, while removing reviews/recommendations from registered users having a fitness metric outside the acceptable threshold to the user fitness metric, so that user does not view the removed reviews. FIG. 7 depicts a modified graphical user interface 165′ of the user device 112 of FIG. 3 showing filtered search results 176′ from the search query 175 for the exercise machine, in accordance with embodiments of the present invention. As shown in FIG. 7, several reviews associated with registered users, or links to reviews of the exercise machine, have been filtered and removed so that the user is unable to select or visually see the links to the reviews written by registered users with an incompatible fitness level. Additionally, the reviews have been ranked and displayed in order of rank based on a lowest deviation of the total fitness metric from the user metric score, as calculated by the computing system 120. For example, Registered User Review 5 is displayed at the top of the results 176′ because the fitness metric score of Registered User 5 has the lowest deviation from the user metric score. Accordingly, reordering the reviews and removing the incompatible reviews may include augmenting a graphical user interface 165 of a user computing device 112 to change an appearance of the graphical user interface 165′ to improve a user purchase decision for user regarding an exercise machine

In alternative embodiments, instead of removing the links from being displayed within the browser, the links may be shown but disabled so that the user knows other reviews from other users came up in the user search query 175, but have been determined to be authored by registered users on a different fitness level than the user. Further, embodiments of the filtering module 134 may present the removed reviews in a separate window along with one or more reasons why the removed review was removed, so that the user can decide whether to read the review with the knowledge that the author is on a different fitness level then the user.

Furthermore, embodiments of the filtering module 134 may validate that the plurality of registered users that have posted a recommendation or review for the exercise machine actually use the exercise machine. For instance, embodiments of the filtering module 134 may analyze the machine usage data of the registered user's exercise machine to determine that the registered user's exercise machine is a same model as the exercise machine, and that the registered user actively uses the registered user's exercise machine. Further, the filtering module 134 may analyze a social media shared content to confirm that the user has indeed used or purchased the exercise machine being purchased by the user. The validation by the filtering module 134 may further increase an accuracy and effectiveness of the review filtering application.

Moreover, embodiments of the filtering module 134 may recommend an ideal exercise machine based on the user fitness metric and the machine data usage obtained from the plurality of internet-connected exercise machines associated with the plurality of registered users. For instance, the filtering module 134 may additionally provide a list of recommended exercise machines that have been suitable for users having a similar fitness metric. Data may be collected and stored by the computing system 120 from various exercise machines 111 to develop insights into the suitability of various exercise machines for users having a given user fitness metric. For example, a user may interact with a computing device at a gym that is running application 130 to decide which equipment to use based on the user's current fitness level. If the user is a beginner or is not accustomed to heavy physical activity, the user may research the machines at the gym and receive feedback/reviews on the machines authored by other users with a corresponding fitness metric score. In another example, the application 130 may be utilized by a physical therapist to select an exercise machine that may work the best for a particular user based on the user's fitness metric score and the historical data received from the machines 111. Further, the data automatically received from the exercise machines 111 may provide insights that can be used to predict a success of an exercise machine for a user with a known user fitness metric. For example, the computing system 120 may determine that how long a user with a given user fitness metric can reach a capacity or max intensity/resistance of the exercise machine, or whether a user with a user fitness metric will actually use certain features associated with more expensive models. Using the stored data received from the exercise machines 111, and continuous profiling of registered users with updated biometric and activity data, the filtering module 134 may generate a recommended exercise machine that has been well-received by users within a closely matched user fitness metric. The recommendation may recommend an exercise machine of a same type as the exercise machine originally searched for by the user, or may recommend a different type of exercise machine than the exercise machine originally searched for by the user.

Various tasks and specific functions of the modules of the computing system 120 may be performed by additional modules, or may be combined into other module(s) to reduce the number of modules. Further, embodiments of the computer or computer system 120 may comprise specialized, non-generic hardware and circuitry (i.e., specialized discrete non-generic analog, digital, and logic-based circuitry) (independently or in combination) particularized for executing only methods of the present invention. The specialized discrete non-generic analog, digital, and logic-based circuitry may include proprietary specially designed components (e.g., a specialized integrated circuit, such as for example an Application Specific Integrated Circuit (ASIC), designed for only implementing methods of the present invention). Moreover, embodiments of the filtering system 100 improves existing search engine technology and an online review/recommendation platform by filtering out links to reviews written by users with a different fitness level than the user searching for a product. Prior search engines and online product review platforms do not intentionally delete results or disable links based on a compatibility of a fitness level of the author with a product being searched and a fitness level of the user. Without the link being present in the search results, the user will not be able to follow a link to make an eventual purchase of an exercise machine that will not be ideal for a user based on the user's fitness level. This reduces product returns and promotes a successful health endeavor of a user so that the user is not discouraged from using a newly purchased exercise machine, or too quickly maximizes the capability of the newly purchased exercise machine. Furthermore, by filtering out the reviews or links associated with users having a different fitness level, the webpage of the user's browser changes in appearance when using the filtering application 130. For example, the filtering system 100 may transform a GUI on a mobile device or other user device of a browser to eliminate mistake in user purchases of exercise machines that may not be suitable for a user given a user's fitness level.

Furthermore, the filtering system 100 improves search engines and online review platforms by solving the problem of presenting exercise machines that are not compatible with the user on a browser for a user to purchase. Without altering the GUI and filtering the initial search results and/or reviews, the de-optimal exercise machines devices are presented to the user. Embodiments of the filtering system 100 provides a technical solution to the above-drawbacks by altering or otherwise augmenting the GUI and filtering the search results to remove links to products a particular user should not purchase based on user fitness level. The technical solution(s) described herein is necessarily rooted in computer technology in order to overcome a problem specifically arising in the realm of search engines and review filtering, based on a fitness level and machine usage data.

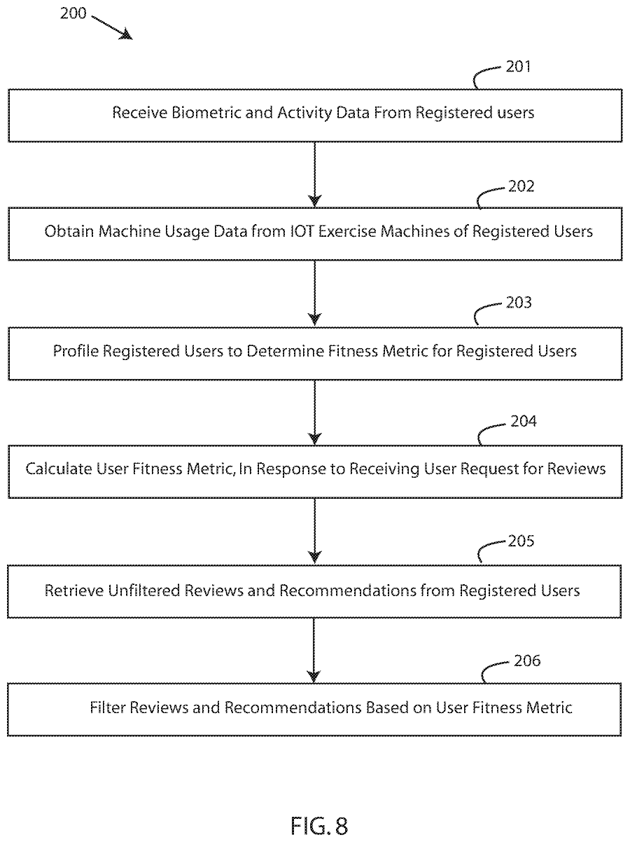

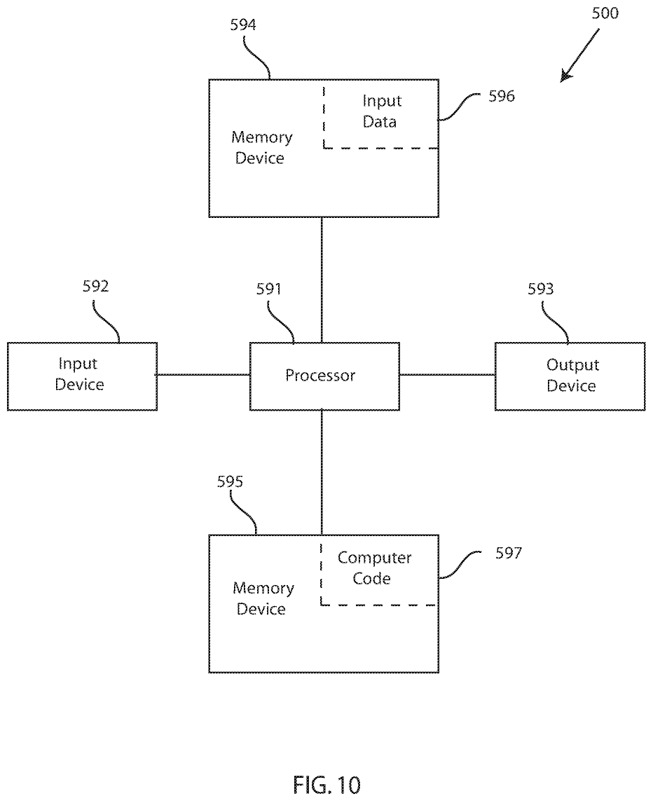

Referring now to FIG. 8, which depicts a flow chart of a method 200 for filtering recommendations for an exercise machine based on a user fitness metric compared to similar fitness metrics of other users, in accordance with embodiments of the present invention. One embodiment of a method 200 or algorithm that may be implemented for filtering recommendations for an exercise machine based on a user fitness metric compared to similar fitness metrics of other users with the filtering system 100 described in FIGS. 1-7 using one or more computer systems as defined generically in FIG. 10 below, and more specifically by the specific embodiments of FIG. 1.

Embodiments of the method 200 for filtering recommendations for an exercise machine based on a user fitness metric compared to similar fitness metrics of other users, in accordance with embodiments of the present invention, may begin at step 201 wherein biometric and activity data is received from registered user device(s). Step 202 obtains machine usage data from IoT exercise machines of registered users. Step 203 profiles registered users to determine a fitness metric for each registered user. Step 204 calculates a user fitness metric, in response to detecting a user inputting a search request for a review of an exercise machine. Step 205 retrieves unfiltered reviews and recommendations from registered users associated with the exercise machine searched by the user. Step 206 filters the reviews and recommendations based on the user fitness metric.

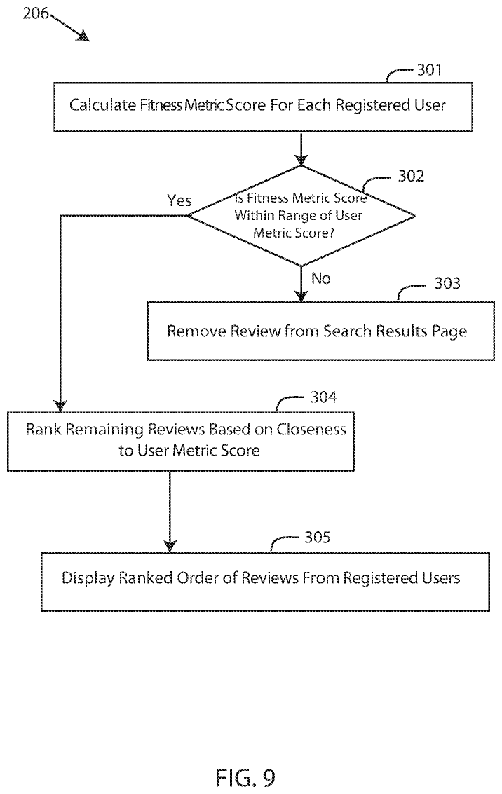

FIG. 9 depicts a flow chart of a step 206 of the method 200 of FIG. 8 for filtering recommendations for an exercise machine based on a user fitness metric compared to similar fitness metrics of other users, in accordance with embodiments of the present invention. Step 301 calculates a total fitness metric score for each registered user. Step 302 determines whether the fitness metric score is within a range of the user metric score. If no, then step 303 removes the reviews or links associated therewith from the webpage. If yes, then step 304 ranks the remaining reviews based on a closeness or deviation from the user metric score. Step 305 displays the ranked order of reviews from the registered users.

FIG. 10 depicts a block diagram of a computer system for the filtering system 100 of FIGS. 1-7, capable of implementing methods for filtering recommendations for an exercise machine based on a user fitness metric compared to similar fitness metrics of other users of FIGS. 8-9, in accordance with embodiments of the present invention. The computer system 500 may generally comprise a processor 591, an input device 592 coupled to the processor 591, an output device 593 coupled to the processor 591, and memory devices 594 and 595 each coupled to the processor 591. The input device 592, output device 593 and memory devices 594, 595 may each be coupled to the processor 591 via a bus. Processor 591 may perform computations and control the functions of computer system 500, including executing instructions included in the computer code 597 for the tools and programs capable of implementing a method for filtering recommendations for an exercise machine based on a user fitness metric compared to similar fitness metrics of other users in the manner prescribed by the embodiments of FIGS. 8-9 using the filtering system 100 of FIGS. 1-7, wherein the instructions of the computer code 597 may be executed by processor 591 via memory device 595. The computer code 597 may include software or program instructions that may implement one or more algorithms for implementing the method for filtering recommendations for an exercise machine based on a user fitness metric compared to similar fitness metrics of other users, as described in detail above. The processor 591 executes the computer code 597. Processor 591 may include a single processing unit, or may be distributed across one or more processing units in one or more locations (e.g., on a client and server).

The memory device 594 may include input data 596. The input data 596 includes any inputs required by the computer code 597. The output device 593 displays output from the computer code 597. Either or both memory devices 594 and 595 may be used as a computer usable storage medium (or program storage device) having a computer-readable program embodied therein and/or having other data stored therein, wherein the computer-readable program comprises the computer code 597. Generally, a computer program product (or, alternatively, an article of manufacture) of the computer system 500 may comprise said computer usable storage medium (or said program storage device).

Memory devices 594, 595 include any known computer-readable storage medium, including those described in detail below. In one embodiment, cache memory elements of memory devices 594, 595 may provide temporary storage of at least some program code (e.g., computer code 597) in order to reduce the number of times code must be retrieved from bulk storage while instructions of the computer code 597 are executed. Moreover, similar to processor 591, memory devices 594, 595 may reside at a single physical location, including one or more types of data storage, or be distributed across a plurality of physical systems in various forms. Further, memory devices 594, 595 can include data distributed across, for example, a local area network (LAN) or a wide area network (WAN). Further, memory devices 594, 595 may include an operating system (not shown) and may include other systems not shown in FIG. 10.

In some embodiments, the computer system 500 may further be coupled to an Input/output (I/O) interface and a computer data storage unit. An I/O interface may include any system for exchanging information to or from an input device 592 or output device 593. The input device 592 may be, inter alia, a keyboard, a mouse, etc. or in some embodiments the touchscreen of a computing device. The output device 593 may be, inter alia, a printer, a plotter, a display device (such as a computer screen), a magnetic tape, a removable hard disk, a floppy disk, etc. The memory devices 594 and 595 may be, inter alia, a hard disk, a floppy disk, a magnetic tape, an optical storage such as a compact disc (CD) or a digital video disc (DVD), a dynamic random access memory (DRAM), a read-only memory (ROM), etc. The bus may provide a communication link between each of the components in computer 500, and may include any type of transmission link, including electrical, optical, wireless, etc.

An I/O interface may allow computer system 500 to store information (e.g., data or program instructions such as program code 597) on and retrieve the information from computer data storage unit (not shown). Computer data storage unit includes a known computer-readable storage medium, which is described below. In one embodiment, computer data storage unit may be a non-volatile data storage device, such as a magnetic disk drive (i.e., hard disk drive) or an optical disc drive (e.g., a CD-ROM drive which receives a CD-ROM disk). In other embodiments, the data storage unit may include a knowledge base or data repository 125 as shown in FIG. 1.

As will be appreciated by one skilled in the art, in a first embodiment, the present invention may be a method; in a second embodiment, the present invention may be a system; and in a third embodiment, the present invention may be a computer program product. Any of the components of the embodiments of the present invention can be deployed, managed, serviced, etc. by a service provider that offers to deploy or integrate computing infrastructure with respect to filtering systems and methods. Thus, an embodiment of the present invention discloses a process for supporting computer infrastructure, where the process includes providing at least one support service for at least one of integrating, hosting, maintaining and deploying computer-readable code (e.g., program code 597) in a computer system (e.g., computer system 500) including one or more processor(s) 591, wherein the processor(s) carry out instructions contained in the computer code 597 causing the computer system to filter recommendations for an exercise machine based on a user fitness metric compared to similar fitness metrics of other users. Another embodiment discloses a process for supporting computer infrastructure, where the process includes integrating computer-readable program code into a computer system 500 including a processor.

The step of integrating includes storing the program code in a computer-readable storage device of the computer system 500 through use of the processor. The program code, upon being executed by the processor, implements a method for filtering recommendations for an exercise machine based on a user fitness metric compared to similar fitness metrics of other users. Thus, the present invention discloses a process for supporting, deploying and/or integrating computer infrastructure, integrating, hosting, maintaining, and deploying computer-readable code into the computer system 500, wherein the code in combination with the computer system 500 is capable of performing a method for filtering recommendations for an exercise machine based on a user fitness metric compared to similar fitness metrics of other users.

A computer program product of the present invention comprises one or more computer-readable hardware storage devices having computer-readable program code stored therein, said program code containing instructions executable by one or more processors of a computer system to implement the methods of the present invention.

A computer system of the present invention comprises one or more processors, one or more memories, and one or more computer-readable hardware storage devices, said one or more hardware storage devices containing program code executable by the one or more processors via the one or more memories to implement the methods of the present invention.

The present invention may be a system, a method, and/or a computer program product at any possible technical detail level of integration. The computer program product may include a computer-readable storage medium (or media) having computer-readable program instructions thereon for causing a processor to carry out aspects of the present invention.

The computer-readable storage medium can be a tangible device that can retain and store instructions for use by an instruction execution device. The computer-readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer-readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer-readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer-readable program instructions described herein can be downloaded to respective computing/processing devices from a computer-readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer-readable program instructions from the network and forwards the computer-readable program instructions for storage in a computer-readable storage medium within the respective computing/processing device.

Computer-readable program instructions for carrying out operations of the present invention may be assembler instructions, instruction-set-architecture (ISA) instructions, machine instructions, machine-dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer-readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer-readable program instructions by utilizing state information of the computer-readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer-readable program instructions.

These computer-readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer-readable program instructions may also be stored in a computer-readable storage medium that can direct a computer, a programmable data processing apparatus, and/or other devices to function in a particular manner, such that the computer-readable storage medium having instructions stored therein comprises an article of manufacture including instructions which implement aspects of the function/act specified in the flowchart and/or block diagram block or blocks.

The computer-readable program instructions may also be loaded onto a computer, other programmable data processing apparatus, or other device to cause a series of operational steps to be performed on the computer, other programmable apparatus or other device to produce a computer-implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

It is to be understood that although this disclosure includes a detailed description on cloud computing, implementation of the teachings recited herein are not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported, providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure that includes a network of interconnected nodes.

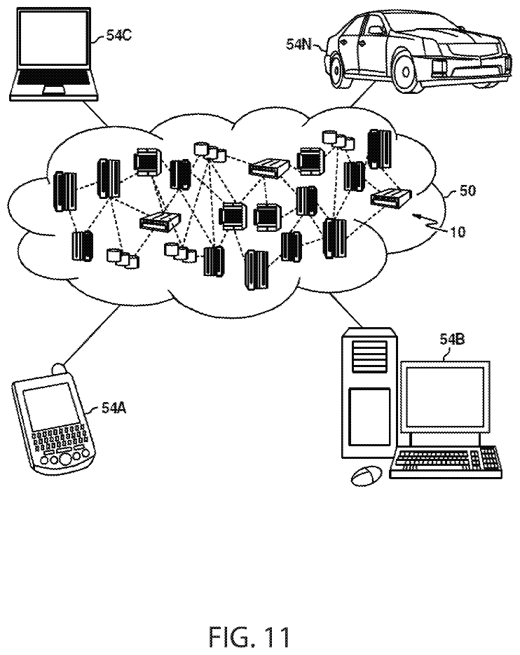

Referring now to FIG. 11, illustrative cloud computing environment 50 is depicted. As shown, cloud computing environment 50 includes one or more cloud computing nodes 10 with which local computing devices used by cloud consumers, such as, for example, personal digital assistant (PDA) or cellular telephone 54A, desktop computer 54B, laptop computer 54C, and/or automobile computer system 54N may communicate. Nodes 10 may communicate with one another. They may be grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain resources on a local computing device. It is understood that the types of computing devices 54A, 54B, 54C and 54N shown in FIG. 11 are intended to be illustrative only and that computing nodes 10 and cloud computing environment 50 can communicate with any type of computerized device over any type of network and/or network addressable connection (e.g., using a web browser).

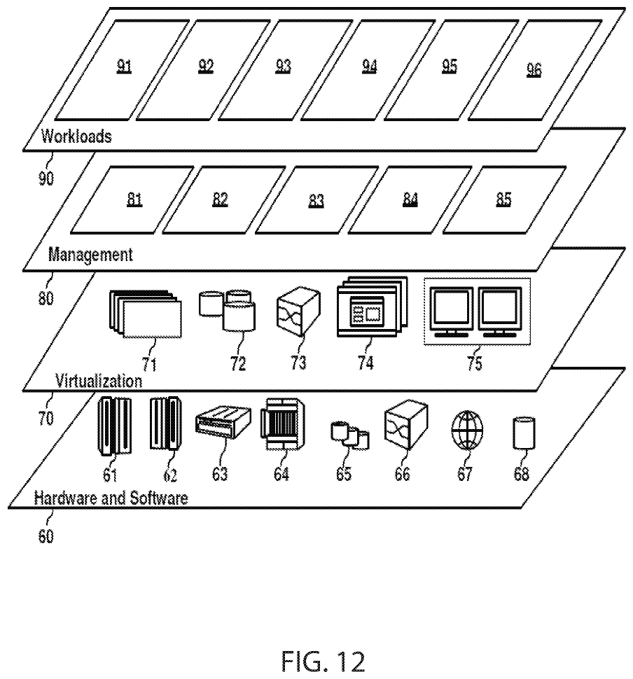

Referring now to FIG. 12, a set of functional abstraction layers provided by cloud computing environment 50 (see FIG. 11) are shown. It should be understood in advance that the components, layers, and functions shown in FIG. 12 are intended to be illustrative only and embodiments of the invention are not limited thereto. As depicted, the following layers and corresponding functions are provided:

Hardware and software layer 60 includes hardware and software components. Examples of hardware components include: mainframes 61; RISC (Reduced Instruction Set Computer) architecture based servers 62; servers 63; blade servers 64; storage devices 65; and networks and networking components 66. In some embodiments, software components include network application server software 67 and database software 68.

Virtualization layer 70 provides an abstraction layer from which the following examples of virtual entities may be provided: virtual servers 71; virtual storage 72; virtual networks 73, including virtual private networks; virtual applications and operating systems 74; and virtual clients 75.

In one example, management layer 80 may provide the functions described below. Resource provisioning 81 provides dynamic procurement of computing resources and other resources that are utilized to perform tasks within the cloud computing environment. Metering and Pricing 82 provide cost tracking as resources are utilized within the cloud computing environment, and billing or invoicing for consumption of these resources. In one example, these resources may include application software licenses. Security provides identity verification for cloud consumers and tasks, as well as protection for data and other resources. User portal 83 provides access to the cloud computing environment for consumers and system administrators. Service level management 84 provides cloud computing resource allocation and management such that required service levels are met. Service Level Agreement (SLA) planning and fulfillment 85 provides pre-arrangement for, and procurement of, cloud computing resources for which a future requirement is anticipated in accordance with an SLA.

Workloads layer 90 provides examples of functionality for which the cloud computing environment may be utilized. Examples of workloads and functions which may be provided from this layer include: mapping and navigation 91; software development and lifecycle management 92; virtual classroom education delivery 93; data analytics processing 94; transaction processing 95; and GUI and filters 96.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein

1. A method for filtering recommendations for an exercise machine based on a user fitness metric compared to similar fitness metrics of other users, the method comprising:
receiving, by a processor of a computing system, biometric and activity data from a plurality of registered users, the biometric and activity data being transmitted from one or more wearable computing devices worn by the plurality of registered users;
obtaining, by the processor, a machine usage data from a plurality of internet-connected exercise machines associated with the plurality of registered users;
profiling, by the processor, the plurality of registered users using the biometric and activity data and the machine usage data to determine a fitness metric of each registered user of the plurality of registered users;
calculating, by the processor, the user fitness metric, in response to receiving a user request for recommendations and reviews for the exercise machine, wherein the user fitness metric is calculated based on a user biometric and activity data transmitted by at least one of a user mobile device and a user wearable device;
retrieving, by the processor, the recommendations and reviews posted by the plurality of registered users relevant to the exercise machine; and
filtering, by the processor, the recommendations and reviews for the exercise machine to exclude recommendations and reviews from the plurality of registered users having a fitness metric that does not correspond to the user fitness metric, such that a website displaying the recommendations and reviews is altered so that the user is unable to view excluded recommendations and reviews for the exercise machine.

2. The method of claim 1, wherein the filtering includes comparing the user fitness metric to fitness metrics of the plurality of registered users that have posted a recommendation and review of the exercise machine, to determine whether the fitness metric of a registered user of the plurality of registered users corresponds to the user fitness metric.

3. The method of claim 2, wherein the fitness metric of the registered user corresponds to the user fitness metric if the fitness metric of the registered user is within a predetermined range of the user fitness metric.

4. The method of claim 1, further comprising validating, by the processor, that the plurality of registered users that have posted a recommendation or review for the exercise machine actually use the exercise machine.

5. The method of claim 4, wherein the validating includes analyzing, by the processor, a machine usage data of the registered user's exercise machine to determine: i) that the registered user's exercise machine is a same model as the exercise machine, and ii) that the registered user actively uses the registered user's exercise machine.

6. The method of claim 1, wherein the profiling includes analyzing, by the processor, a shared social media content of the plurality of registered users to augment the fitness metric of the plurality of registered users.

7. The method of claim 1, further comprising recommending, by the processor, an ideal exercise machine based on the user fitness metric and the machine data usage obtained from the plurality of internet-connected exercise machines associated with the plurality of registered users.

8. A computing system, comprising:
a processor;
a memory device coupled to the processor; and
a computer readable storage device coupled to the processor, wherein the storage device contains program code executable by the processor via the memory device to implement a method for filtering recommendations for an exercise machine based on a user fitness metric compared to similar fitness metrics of other users, the method comprising:
receiving, by a processor of a computing system, biometric and activity data from a plurality of registered users, the biometric and activity data being transmitted from one or more wearable computing devices worn by the plurality of registered users;
obtaining, by the processor, a machine usage data from a plurality of internet-connected exercise machines associated with the plurality of registered users;
profiling, by the processor, the plurality of registered users using the biometric and activity data and the machine usage data to determine a fitness metric of each registered user of the plurality of registered users;
calculating, by the processor, the user fitness metric, in response to receiving a user request for recommendations and reviews for the exercise machine, wherein the user fitness metric is calculated based on a user biometric and activity data transmitted by at least one of a user mobile device and a user wearable device;
retrieving, by the processor, the recommendations and reviews posted by the plurality of registered users relevant to the exercise machine; and
filtering, by the processor, the recommendations and reviews for the exercise machine to exclude recommendations and reviews from the plurality of registered users having a fitness metric that does not correspond to the user fitness metric, such that a website displaying the recommendations and reviews is altered so that the user is unable to view excluded recommendations and reviews for the exercise machine.

9. The computing system of claim 8, wherein the filtering includes comparing the user fitness metric to fitness metrics of the plurality of registered users that have posted a recommendation and review of the exercise machine, to determine whether the fitness metric of a registered user of the plurality of registered users corresponds to the user fitness metric.

10. The computing system of claim 9, wherein the fitness metric of the registered user corresponds to the user fitness metric if the fitness metric of the registered user is within a predetermined range of the user fitness metric.

11. The computing system of claim 8, further comprising validating, by the processor, that the plurality of registered users that have posted a recommendation or review for the exercise machine actually use the exercise machine.

12. The computing system of claim 11, wherein the validating includes analyzing, by the processor, a machine usage data of the registered user's exercise machine to determine: i) that the registered user's exercise machine is a same model as the exercise machine, and ii) that the registered user actively uses the registered user's exercise machine.

13. The computing system of claim 8, wherein the profiling includes analyzing, by the processor, a shared social media content of the plurality of registered users to augment the fitness metric of the plurality of registered users.

14. The computing system of claim 8, further comprising recommending, by the processor, an ideal exercise machine based on the user fitness metric and the machine data usage obtained from the plurality of internet-connected exercise machines associated with the plurality of registered users.

15. A computer program product, comprising a computer readable hardware storage device storing a computer readable program code, the computer readable program code comprising an algorithm that when executed by a computer processor of a computing system implements a method for filtering recommendations for an exercise machine based on a user fitness metric compared to similar fitness metrics of other users, the method comprising:
receiving, by a processor of a computing system, biometric and activity data from a plurality of registered users, the biometric and activity data being transmitted from one or more wearable computing devices worn by the plurality of registered users;
obtaining, by the processor, a machine usage data from a plurality of internet-connected exercise machines associated with the plurality of registered users;
profiling, by the processor, the plurality of registered users using the biometric and activity data and the machine usage data to determine a fitness metric of each registered user of the plurality of registered users;
calculating, by the processor, the user fitness metric, in response to receiving a user request for recommendations and reviews for the exercise machine, wherein the user fitness metric is calculated based on a user biometric and activity data transmitted by at least one of a user mobile device and a user wearable device;
retrieving, by the processor, the recommendations and reviews posted by the plurality of registered users relevant to the exercise machine; and
filtering, by the processor, the recommendations and reviews for the exercise machine to exclude recommendations and reviews from the plurality of registered users having a fitness metric that does not correspond to the user fitness metric, such that a website displaying the recommendations and reviews is altered so that the user is unable to view excluded recommendations and reviews for the exercise machine

16. The computer program product of claim 15, wherein the filtering includes comparing the user fitness metric to fitness metrics of the plurality of registered users that have posted a recommendation and review of the exercise machine, to determine whether the fitness metric of a registered user of the plurality of registered users corresponds to the user fitness metric.

17. The computer program product of claim 16, wherein the fitness metric of the registered user corresponds to the user fitness metric if the fitness metric of the registered user is within a predetermined range of the user fitness metric.

18. The computer program product of claim 15, further comprising validating, by the processor, that the plurality of registered users that have posted a recommendation or review for the exercise machine actually use the exercise machine.

19. The computer program product of claim 18, wherein the validating includes analyzing, by the processor, a machine usage data of the registered user's exercise machine to determine: i) that the registered user's exercise machine is a same model as the exercise machine, and ii) that the registered user actively uses the registered user's exercise machine.

20. The computer program product of claim 15, further comprising recommending, by the processor, an ideal exercise machine based on the user fitness metric and the machine data usage obtained from the plurality of internet-connected exercise machines associated with the plurality of registered users.
